(19) United States
(12) Patent Application Publication
(10) Pub. No.: US 2007/0123792 A1
(43) Pub. Date: May 31, 2007

(54) System and method for determining airway obstruction

(75) Inventor: Jeffrey A. Kline, Charlotte, NC (US)

Correspondence Address: BOND, SCHOENECK & KING, PLLC, ONE LINCOLN CENTER, SYRACUSE, NY, 13202-1355, US

(73) Assignee: Charlotte-Mecklenburg Hospital Authority d/b/a Carolinas Medical Center, Charlotte, NC (US)

(21) Appl. No.: 11/282,012

(22) Filed: Nov. 17, 2005

(51) Int. Cl. A61B 5/08; A61B 5/00

(52) U.S. Cl. 600/538; 600/529; 600/323

(57) ABSTRACT

A method and system for detecting the presence of restriction to expired airflow in humans or animals by analyzing the expired capnogram and oxygram, as well as the geometric analysis of the real-time plot of the waveform that depicts the instantaneous ratio of CO₂ to O₂ (the carboxygram ratio). Airway obstructions causes an increase in the Q-angle between the slope of phase 11 and slope of phase III in the expired carboxygram. The diagnostic accuracy of the detection of airways obstruction is further enhanced by measuring the ratio of time spent in exhalation (Te) versus inhalation (Ti). The system uses the combination of an increased carboxygram Q-angle, and a prolonged Te/Ti to detect presence of airways obstruction.

### BACKGROUND OF THE INVENTION

1. Field of Invention

The present invention relates to the diagnosis of airways obstruction and, more specifically, to a system and method for determining the severity and cause of breathing difficulties in respiratory patients.

2. Description of Prior Art

Obstruction of the breathing passages within the lungs represents a common medical condition. Approximately 20 million Americans have the condition of bronchial asthma, and another 7 million have the condition of chronic obstructive pulmonary disease (COPD). Several million other Americans have intermittent spells of difficulty breathing caused by reversible airways hyperreactivity. While the underlying causes of all of these conditions differ, they all produce restriction to airflow during exhalation.

In human and veterinary medicine, clinicians measure the severity of airways restriction to guide treatment decisions. In human medicine, the severity of restriction is quantified by currently measuring the maximal rate of airflow during a forced exhalation. The most common embodiments of this method include the forced exhalation volume during one second (FEV₁) and the peak flow measurement. The measurement of peak exhaled airflow requires the patient to hold a mouthpiece with an airtight seal, and to exhale rapidly and forcefully as possible. This process inherently incorporates an unquantifiable variable of patient cooperation. Accordingly, abnormally low readings are often unreliable, especially in acutely ill patients.

### OBJECTS AND ADVANTAGES

It is a principal object and advantage of the present invention to provide a system and method for determining the presence and severity of airways obstruction.

It is a further object and advantage of the present invention to provide a system and method for measuring the presence and severity of airways obstruction that is less effort-dependent.

It is an additional object and advantage of the present invention to provide a system and method for measuring the presence and severity of airways obstruction that is more reliable.

It is also an object and advantage of the present invention to provide a system and method for measuring the presence and severity of airways obstruction that is easier to reproduce in home and clinical settings.

Other objects and advantages of the present invention will in part be obvious, and in part appear hereinafter.

### SUMMARY OF THE INVENTION

The present invention comprises a system and method for simultaneously measuring the pCO₂ and pO₂ of a patients and plotting of the ratio of CO₂/O₂ instantaneously (hereinafter referred to as the “carboxygram”) to determine whether the shape of the carboxygram has been deformed in manner indicative of airways obstruction. The effect of an airways obstruction on the expired oxygram and carboxygrams, i.e., the tracing of the partial pressure of expired oxygen (pO₂) and the partial pressure of expired carbon dioxide (pCO₂), will deform in a predictable manner. The system and method of the present invention measures partial pressures of expired oxygen and carbon dioxide and then determines the effect of airways obstruction on both the capnogram and the oxygram to diagnose and/or predict the presence an airways obstruction in a patient. The system and method of the present invention also uses the delay in the time period required for expiration (Te) compared with inspiration (Ti) to diagnose airways obstruction. Based on the results of the measurements taken according to the present invention, a preliminary diagnosis may be reached by comparing the measured results to normal and afflicted populations.

### BRIEF DESCRIPTION OF THE DRAWINGS

The present invention will be more fully understood and appreciated by reading the following Detailed Description in conjunction with the accompanying drawings, in which:

FIG. 1 is a graph of a capnogram according to the present invention.

FIG. 2 is a graph of a capnogram according to the present invention.

FIG. 3 is a graph of a capnogram according to the present invention.

FIG. 4 is a graph of a capnogram according to the present invention.

FIG. 5 is a schematic of a system according to the present invention.

FIG. 6 is a schematic of another embodiment according to the present invention.

FIG. 7 is a schematic of a further embodiment according to the present invention.

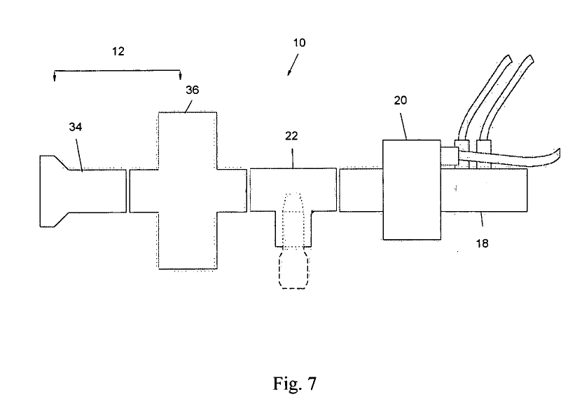

FIG. 8 is an example of a visual display of a system according to the present invention.

FIG. 9 is an example of a visual display of a system according to the present invention.

FIG. 10 is an example of a visual display of a system according to the present invention.

FIG. 11 is an example of a visual display of a system according to the present invention.

FIG. 12 is an example of a visual display of a system according to the present invention.

FIG. 13 is an example of a visual display of a system according to the present invention.

FIG. 14 is a graphical comparison of measurements according to the present invention.

FIG. 15 is a graph of a carboxygram according to the present invention.

FIG. 16 is a graph of a carboxygram according to the present invention.

FIG. 17 is a graph of the results of a calculation according to the present invention.

FIG. 18 is a graph of the results of a calculation according to the present invention.

### DETAILED DESCRIPTION

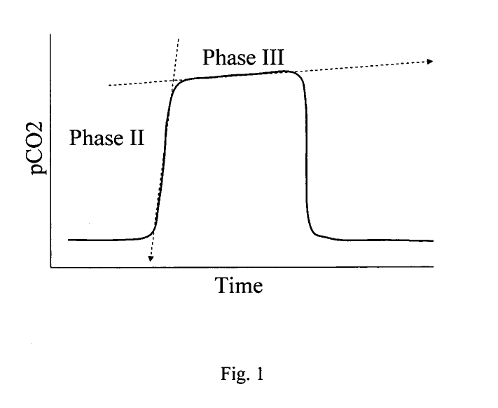

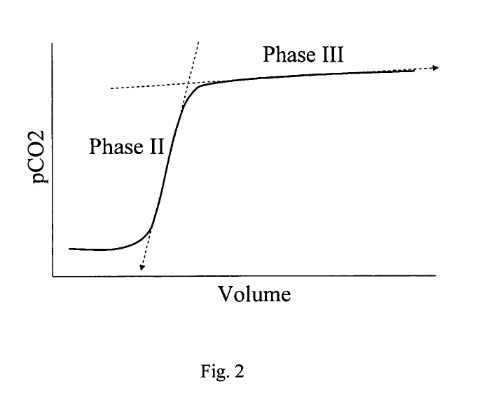

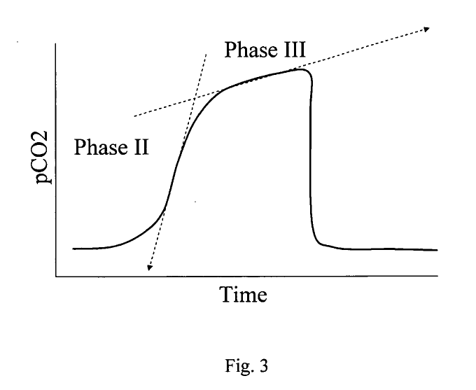

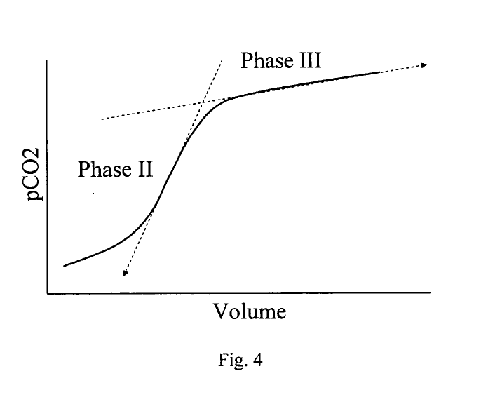

Referring now to the drawings, wherein like numerals refer to like parts throughout, there is seen in FIGS. 1 and 2 two graphs depicting the measurement of expired PCO₂ (capnograms) as a function of time and volume, respectively, in a normal patient. A patient having an acute airways obstruction will demonstrate altered geometry of these curves, as seen in FIGS. 3 and 4. The two components of the expired capnogram that are affected by airways obstruction are the slopes of the plotted curves in the areas designated as Phase II and Phase III. Phase II represents the volume of breath that empties the conducting airways of the lungs, including the trachea and bronchioles. This volume is collectively termed the airway or anatomic deadspace portion of expired breath. Phase III depicts the partial pressure of CO₂ (pCO₂) contained within gas expired from the alveoli. As seen in FIGS. 1 and 2, the slope (or first derivative) of Phase II is generally high in normal patients. The high slope depicts the normal sharp and rapid transition that occurs as the conducting airways empty their content of ambient air, and begin to expire alveolar gas which was equilibrated with the CO₂ content in mixed venous blood. Conversely, the slope of Phase III is flat, representing a relatively homogenous partial pressure of CO₂ within alveolar gas.

Referring to FIGS. 3 and 4, the sharp and rapid transition described in phase II becomes blunted in patients with disease-induced restriction to expired airway flow. With disease, a proportion of airways remain patent, while a proportion is partially or totally occluded. During exhalation, the patent airways empty first, and begin to transition to the alveolar portion of the breath, while in the partially occluded airways the transition to the alveolar phase is delayed. As a result, alveolar gas from the patent airways mixes with the anatomic gas from the diseased airways, contributing to an increased amount of CO₂ in Phase II of the curve, causing its slope to decrease. This non-homogenous emptying also affects Phase II, because the restricted airways require variable time periods to empty. This variable time requirement causes two effects that contribute to the increased slope of Phase III. The first is the continued heterogenous mixing of conducting gas with alveolar gas, and the second is an increase in the time needed for the alveolar gas to equilibrate with the mixed venous blood in the most diseased airways, resulting in higher pCO₂ in the expired gas that is the most delayed.

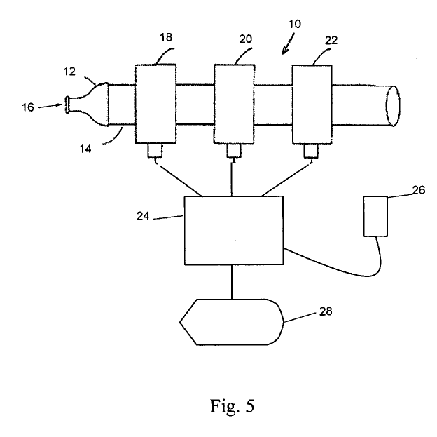

Referring to FIG. 5, the present invention includes a device 10 for measuring the volume air and PCO₂ and pO₂ expired from a patient. Device 10 comprises a patient mouthpiece 12 connected in fluid communication to a breathing tube 14 having an open end 16 through which air may be exhaled or inhaled by a patient. Device 10 further comprises a airflow transducer or pneumotach 18 for measuring expired flow rate, a fast-response sensor 20 for measuring CO₂ and a fast response sensor 22 for measuring 02, all of which are situated in series and in-line with breathing tube 14 for simultaneously measuring the flow, carbon dioxide, and oxygen levels of air inhaled and exhaled by a patient through the tube. Pneumotach 18, carbon dioxide sensor 20, and oxygen sensor 22 are electrically interconnected to a microprocessor 24 having an analog-to-digital converter for sampling the electrical outputs of the measuring elements. Device 10 further comprises a pulse oximetry module 26 electrically interconnected to microprocessor 24. Microprocessor 24 is electrically interconnected to a screen 28 for visually displaying various calculations, measurements, and graphical representations of the measured data according to the present invention.

Microprocessor 24 should be programmed to provide a Ti/Te ratio and calculate the slope of graph of the CO2/O2 ratios during Phase II and Phase III of the running carboxygram plot. Microprocessor 24 may comprise a MP100 system available from Biopac Systems, Inc, of Santa Barbara, Calif. Microprocessor 24 must determine the running average of Ti and Te and compute the average Ti/Te based upon the mean value obtained from breaths obtained during approximately a 30 second period of breathing. This value can be displayed as “summary data” on screen 28. Screen 28 can also provide reference intervals for Ti/Te, as measured in healthy subjects and patients with various disease states, including diseases that cause airway obstruction, and pulmonary embolism to assist in clinical diagnosis. For example, patients diagnosed with pulmonary embolism have a mean Ti/Te of 0.72±0.13, patients having had pulmonary embolism ruled out have a mean Ti/Te of 0.71±0.26, healthy patients have a Ti/Te of 0.75±0.15, and patient with acute exacerbation of bronchial asthma have a Ti/Te of 0.45±0.35.

Microprocessor 24 should also be programmed to normalize the signals obtained for all sensors to correct for differential sensor speed. For example, in general, oxygen sensing devices require more time to respond to a change in oxygen partial pressure, compared with the ability of an infrared absorption detection system to respond to a change in partial pressure of carbon dioxide. If at a given flow rate, an oxygen sensor has a delay of 250 ms, and a carbon dioxide sensor which has a delay of 50 ms (both sensors operating at the same frequency), then microprocessor 24 must match any given CO₂ data point with an O₂ data point that arrives 200 ms later. Microprocessor 24 must execute this delay correction according to differential sensor delays as a function of flow rate.

Microprocessor 24 should also be programmed to determine the slopes of Phase II and III of the carboxygrams obtained from the two deep exhalations and the average slopes obtained during 30 seconds of tidal breathing. These slopes can be computed with two X-axes; time and volume. To facilitate clinician understanding, microprocessor 24 should be programmed to report the overlay of several breaths obtained during a 30 second period of tidal breathing, plotting the CO₂/O₂ ratio as a function of either time or volume.

Carbon dioxide and oxygen partial pressures may be quantified in real-time by sensors 20 and 22 that are capable of performing infrared absorptiometry and paramagnetic deviation, respectively. An acceptable absorptiometer sensor 20 is Model No. C02100C Carbon Dioxide Measurement Model available from Biopac Systems, and an acceptable paramagnetic sensor 22 is Model No. 02100C Oxygen Measurement Module, also available from Biopac Systems. Sensors 20 and 22 should be calibrated against two dry reference gases (0% CO₂/21% O₂ and 7.5% CO₂/12% O₂) before sampling from a patient, and the readings of the reference gases should be repeated immediately after data is collected from each patient to evaluate for calibration stability.

Airflow transducer 18 should be tested against a volumetric calibration syringe, such as Model No. AFT 26 2L, available from Biopac Systems, immediately before and after each patient. Airflow, expired volume, continuous tracings of expired CO₂ and O₂ are recorded at body temperature and saturated with water and at ambient pressure (BTSP). The data may be archived digitally after analog-to-digital conversion by using commercially available software, such as the ACK100W AcqKnowledge software available from Biopac Systems.

Mouthpiece 12 into which the patient breathes can comprise a standard plastic duckbill mouthpiece where the patient forms a seal against the device, a rubber bit-block device that the patient puts into his or her mouth, or a face mask as described next. Examples of such devices may be commonly found in conventional respiratory therapy supply carts, such as a Hudson RCI plastic duckbill, a rubber Kraton ⅞″ internal diameter, reusable mouthpiece (Catalog No. 1645 of AM Systems, Inc. of Carlsborg, Wash.), or a Hans Rudolph series 7600 full face mask with three-way valve to allow measurement of the partial pressure of therapeutic oxygen and the partial pressure of oxygen in expired breath. The latter configuration is especially desirable in a patient with severe respiratory distress to allow delivery of exogenous oxygen and to measure the inspired pO₂ and expired pO₂. Other full face masks are equally adaptable for use in connection with the present invention, including the disposable Mirage mask available from ResMed Ltd. of Sydney NSW, Australia.

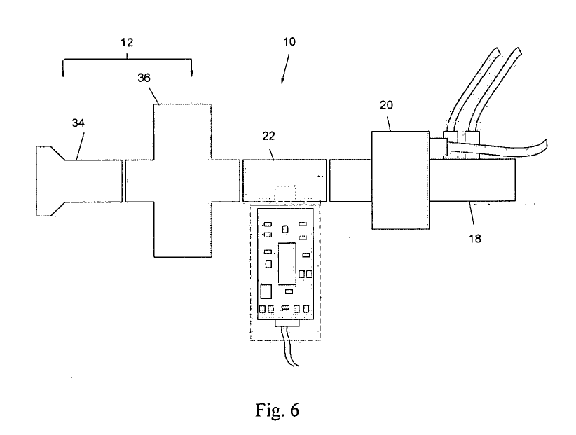

Referring to FIG. 6, mouthpiece 12 may be a disposable assembly of a first portion 34 coupled with a dehumidifying chamber 36. In an alternative embodiment, disposable portion 34 includes a portion of O₂ sensor 22. For either embodiment, the various O₂, CO₂ and flow sensors, are preferably are lightweight (<100 grams in total), compact, and have fast response times (<50 ms). In addition, the deadspace volume should be not more than 15 mL, and the inner diameter should be approximately 13 mm. Each end of device 10 should further be adaptable to couple with an endotracheal tube to allow connection within a ventilatory circuit for use with a patient receiving mechanical ventilation.

Oxygen sensor 22 can operate using known principles of detection such as galvanic, paramagnetic, mass- or laser-spectrometry, calorimetry, or fluorescent detection. Commercially available oxygen sensors include the electrochemical sensor manufactured by Sensors for Medicine and Science, Inc. of Germantown, Md. (http://www.s4ms.com) or the fluorescent sensor known as the SentrOxy OEM-PFT available through Sentronic GmbH (http://www.sentronic.net).

Carbon dioxide sensor 20 can operate using either non-dispersive infrared absorption, mass- or laser-spectrometric detection. A commercially available CO₂ sensor suitable to this purpose is the Capnostat mainstream etCO₂ infrared sensor available from Respironics, Wallingford, Conn. Multiple methods can be used to detect mainstream flow, including those that employ Bernoulli's equation based upon pressure differential across an orifice, those that use thermal differential methods, and those that use piezieolectric principles.

Flow sensor 18 should have a detection range from zero to a minimum of 15 L/Sec with an accuracy of approximately ±3%. A commercially available device that meets these tolerances is the Vmax mass flow sensor available from SensorMedics, Yorba Linda, Calif. Flow data can then be integrated to yield volume. Although these particular measuring technologies represent an acceptable means for detecting O₂, CO₂ and flow, it should be recognized by one of skill in the art that other technologies could be employed to achieve the same objective.

Each sensor 18, 20, and 22 produces an electrical current that is digitized by microprocessor 24 prior to analysis by using an analog-to-digital converter with sufficient bandwidth and a sampling rate of aproximately 75 Hz to 300 kHz. Microprocessor 24 must perform basic functions for measuring Ti and Te and computing the average Ti/Te for a present period of breath collection (e.g., one minute).

The configuration of sensors 18, 20, and 22 can affect the device performance. In the preferred embodiment, the flow sensor 12, CO₂ sensor 20, and O₂ sensor 22 are positioned in a mainstream fashion to measure each parameter directly within the path of exhaled breath, as seen in FIGS. 5 and 6. As an alternative, measurement of CO₂ and O₂ to occur may be taken in sidestream by transferring sample air via vacuum tubing to the applicable sensors. This embodiment, while theoretically feasible, is less desirable due to the difficulty of compensating for errors introduced by the variables such as the rate of vacuum aspiration, tubing length, diameter, condensation, tubing kinking, and other problems.

According to the method of the present invention, device 10 is provided to a patient for measurement of the various gases. The patient should breathe ambient air for at least two minutes prior to taking measurements with device 10. Breaths are collected from a patient seated in semi-Fowler's position and wearing nose clips. Patients should deliver a sharp, rapid, deep exhalation to a maximum endpoint, starting from a midpoint of tidal breathing (i.e., not delivered after a sigh inspiration), followed by a few normal breaths, and then a thirty second period of tidal breathing. All measurements may be taken during this breath collection interval. This sequence should be repeated twice more, yielding three deep exhalations and three 30-second samples of tidal breathing.

Cooperative patients can hold device 10 in their hands, and breathe into mouthpiece 12. The patient should first provide a deep exhalation, and then breathe for 30 seconds, followed by a second deep exhalation. All measurements may be taken during this breath collection interval. For obtunded patients or those with severe distress, breaths can be collected using a face mask connected in fluid series to a T-piece with valves oriented to allow oxygen to be delivered such that both the inspiratory and expiratory pO₂ can be measured.

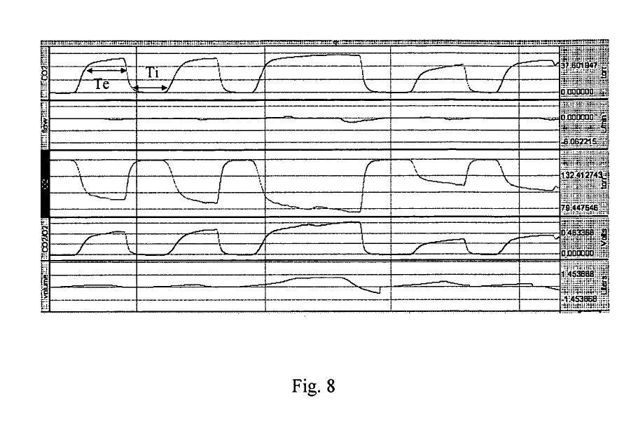

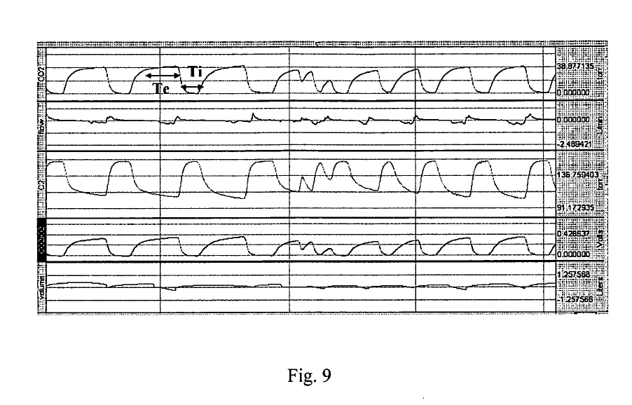

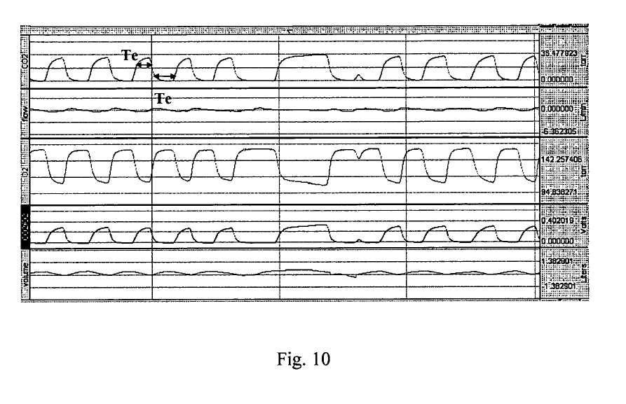

FIGS. 8, 9, and 10 depict measurements obtained during spontaneous breathing from a healthy control subject, a subject with airway obstruction from bronchial asthma, and a subject with pulmonary embolism, respectively, according to the procedures detailed above. FIGS. 8, 9, and 10 demonstrate that the Te is generally prolonged relative to Ti in the patient with bronchial asthma. FIGS. 8, 9, and 10 also show that the Te and Ti may be deduced from the capnogram, but it should be obvious that Te and Ti could be estimated from other measured or calculated parameters including expired flow, volume, pO₂, the ratio of pCO₂/pO₂, or pN₂.

FIGS. 8, 9, and 10 further illustrate that the expiratory capnograms, oxygrams and the carboxygrams differ between normal patients, patients with asthma, and patients with pulmonary embolism. In particular, normal patients have capnograms and carboxygrams with a larger area under each curve, but with fewer breaths per unit of time compared with either patients with asthma or patients with pulmonary embolism. Patients with pulmonary embolism demonstrate capnograms and carboxygrams with particularly small areas.

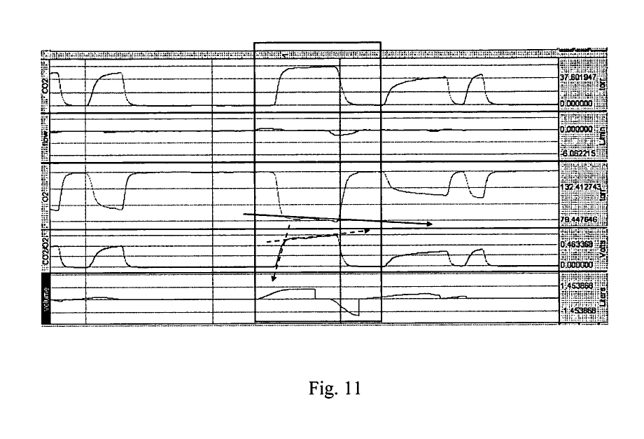

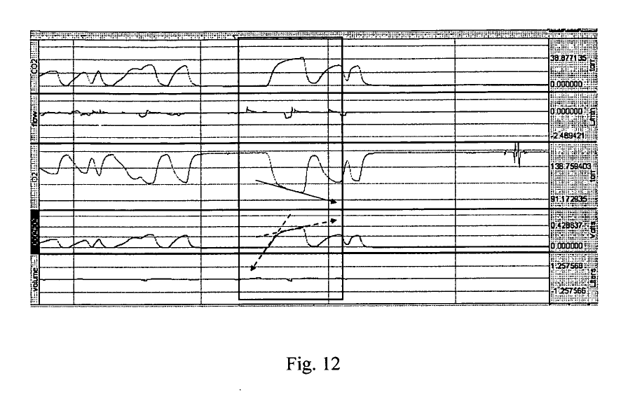

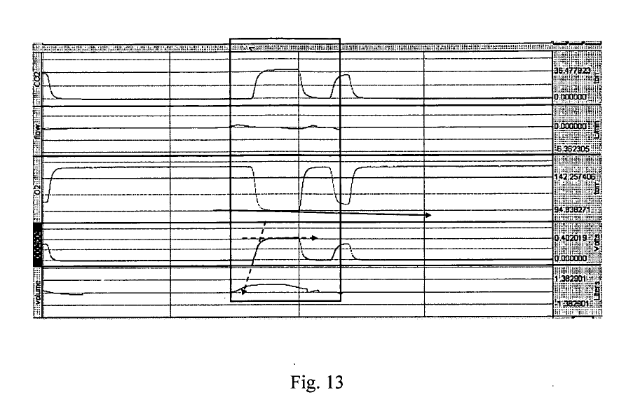

There is seen in FIGS. 11, 12, and 13, plots of a breath obtained from a single deep exhalation illustrate the effect of an airways obstruction on the expired oxygram and carboxygram. FIG. 11 was obtained from a normal subject, FIG. 12 from a patient with acute asthma, and FIG. 13 from a patient with pulmonary embolism. The arrows drawn under the nadir asymptote of the boxed-in oxygram for each patient represent a visual estimation of the first derivative of this asymptote. This portion of the oxygram corresponds to Phase III of the capnogram. It can be seen that the slope of the Phase III portion of the oxygram increases in a patient with asthma.

In FIGS. 11, 12, and 13, the fourth tracing illustrates the carboxygram (instantaneous ratio of CO₂:O₂). The dotted arrows in FIGS. 11 and 12 are drawn approximately tangent to the Phase III component, and illustrate an increase in slope in the patient with asthma. Similarly, the slope of Phase II is decreased only in the patient with asthma.

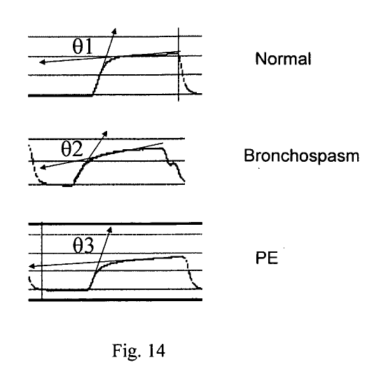

Referring to FIG. 14, three representative carboxygrams from FIGS. 11, 12, and 13 are reproduced for comparison and analysis. The Q-angle is denoted by 01 for a normal subject, θ2 for the patient a bronchial asthma, and θ3 for a patient with pulmonary embolism. The graphs show that θ2 is widened more than θ1 or θ3. The measurement of these angles in normal subjects is a mean of 110±8 degrees, in patients with asthma is a mean of 132±4 degrees, and in patients with PE is a mean of 105±5 degrees. In general, patients with clinically significant airways restriction demonstrate a θ greater than 120°.

Inspiratory time, Ti can be defined by the resulting capnogram, the oxygram, or the flow data. Using flow curves to define the start and stop of Ti and Te provides a theoretical advantage of estimating the start of exhalation during the initial emptying phase of the airways and before CO₂ increases and O₂ decreases. On the other hand, CO₂ increases and O₂ decreases during exhalation only after the airway deadspace (100-300 mL) has mostly evacuated and the subject begins to empty the alveoli. Typically, dual thresholds in flow are used to mark the start of exhalation and inhalation, including a >±10 L/min rate of flow change, and greater than 25 mL total volume change in an adult. Similarly, the Ti and Te can be marked by the true upslope of the CO₂ curve (based upon a trigger consisting of an absolute CO₂ value >2.0 mm Hg and a +10 mm Hg CO₂/sec rate of rise) and return to the baseline, using similar values. Likewise, thresholds can be set on the oxygram upslope and downslope to mark the start of exhalation and inhalation, respectively.

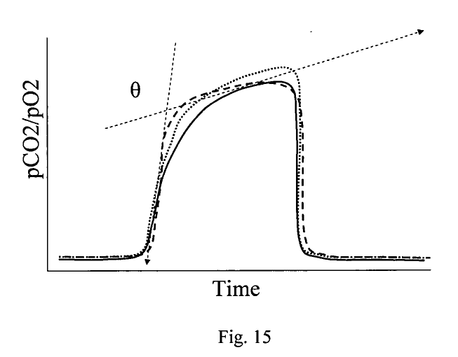

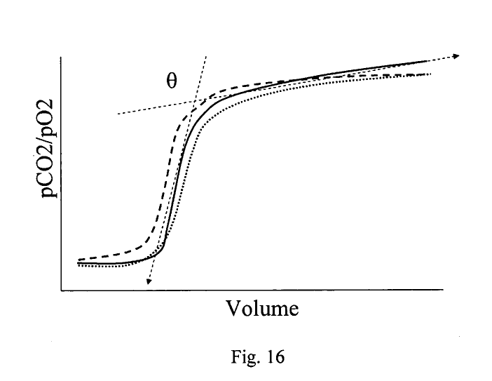

FIGS. 15 and 16 schematically demonstrate three different carboxygrams from three breaths as three different lines; one with short dashes, a second with long dashes and a third via a solid line. The dashed straight lines represent the average value of the vectors defined by Phases II and III for each of the three carboxygrams. Microprocessor 24 may also produce an output to screen 28 to display that demonstrate the best-fit slope of phase II and phase III and that report the mean θ. These values are also exported in numeric format (with mean and variance data as needed) to screen 28. Screen 28 then reports the values of each variable measured in previously studied cohorts of normal subjects and patients with airway restriction and patients with pulmonary embolism.

In an alternative embodiment, microprocessor 24 is programmed to instantly differentiate the change in the ratio of CO₂/O₂ as a function of time or volume according to the equations, where t=time and V=expired breath volume:
F(x)=d(CO₂/O2)/dt
F(x)=d(CO2/O2)/dV

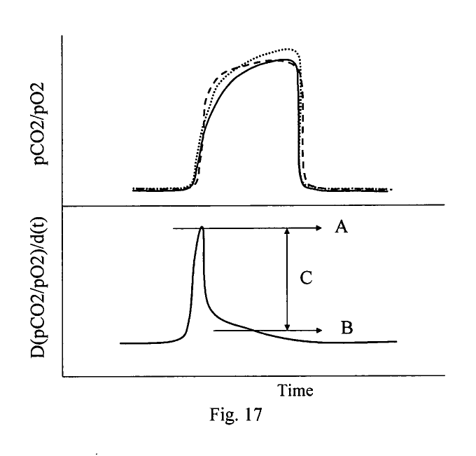

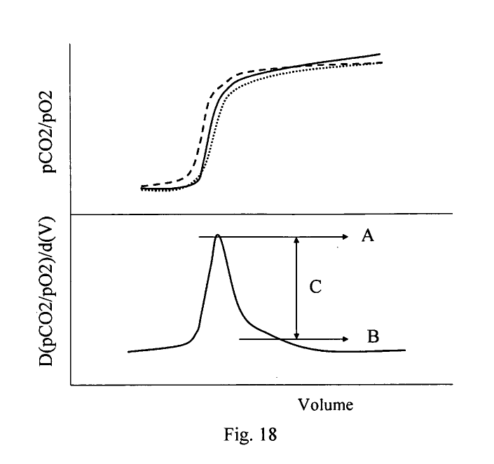

FIGS. 17 and 18 illustrate an output according to this embodiment. In this case, the maximum positive deflection A represents the slope of phase II, and the mean value of the descending flat portion B represents the slope of phase III. The difference C, obtained by subtracting B from A, varies directly in proportion to θ. The numeric values of A, B and their difference C may be exported and shown on screen 28 as a summary page or depicted relative to previously measured data in normal and diseased subjects.

Although the present invention focuses on the analysis of a carboxygram, it should be obvious to those skilled in the art that other gases could be used to measure the severity of airway restriction, including a plot of pN₂ or plots of ratios containing pN₂ as a numerator or denominator. Likewise, the device could be configured to detect similar changes in slope of the partial pressure of exogenously inhaled and poorly absorbed gases, including inert gases such as helium.

What is claimed is:

1. A system for diagnosing the presence of abnormal respiratory function, comprising: ‘a breathing tube through which a subject may take one or more breaths over a predetermined time period;
a flow meter connected to said tube;
an oxygen meter connecter to said tube;
a carbon dioxide meter connected to said tube; and
a microprocessor connected to said flow meter, said oxygen meter, said carbon dioxide meter, and said pulse oximeter, wherein said microprocessor is programmed to calculate the ratio of carbon dioxide to oxygen in said breaths in real-time.

2. The system of claim 1, wherein the microprocessor is programmed to correct for the differential response rates of carbon dioxide meters.

3. The system of claim 1, further comprising a display screen connected to said microprocessor.

4. The system of claim 3, wherein said display screen displays a plot of the ratios of the carbon dioxide to oxygen in real-time over said predetermined time period.

5. The system of claim 4, wherein said display screen displays the plot of the ratios of carbon dioxide to oxygen as a smoothed line.

6. The system of claim 3, wherein said display screen displays a plot of the ratios of the partial pressures of carbon dioxide to oxygen in real-time over a predetermined time period in combination with previously measured ratios of partial pressures of carbon dioxide to oxygen in normal and afflicted populations.

7. The system of claim 3, wherein said microprocessor is programmed to calculate a running average inspiration time and expiration time over a predetermined time period.

8. The system of claim 7, wherein said display screen displays a plot of the running average inspiration time and expiration time over a predetermined time period.

9. The system of claim 8, wherein said display screen displays the plot of the running average inspiration time and expiration time over a predetermined time period as a smooth line.

10. The system of claim 8, wherein said display screen displays a plot of the running average inspiration time and expiration time over a predetermined time period in combination with previously measured average inspiration time and expiration time in normal and afflicted populations.

11. The system of claim 3, wherein said microprocessor is programmed to calculate the straight line slope of a first predetermined portion of a plot of the ratio of carbon dioxide to oxygen.

12. The system of claim 11, wherein said microprocessor is programmed to calculate the straight line slope of a second predetermined portion of a plot of the ratio of carbon dioxide to oxygen.

13. The system of claim 12, wherein said microprocessor is programmed to determine the widest angle formed by the intersection of the straight line slope of the first predetermined portion and the straight line slope of the second predetermined portion.

14. The system of claim 3, wherein said microprocessor is programmed to calculate the first derivative of a first predetermined portion of a plot of the ratio of carbon dioxide to oxygen.

15. The system of claim 14, wherein said microprocessor is programmed to calculate the first derivative of a second predetermined portion of a plot of the ratio of carbon dioxide to oxygen.

16. The system of claim 15, wherein said microprocessor is programmed to calculate the difference in the maximum first derivative of the first predetermined portion minus the maximum first derivative of the second predetermined portion.

17. The system of claim 4, further including a database containing previously measured ratios of carbon dioxide to oxygen in normal and afflicted subjects interconnected to said microprocessor.

18. The system of claim 17, wherein said display screen displays the plot of the ratios of the carbon dioxide to oxygen in combination with a plot of the previously measured ratios of carbon dioxide to oxygen in normal and afflicted subjects.

19. A method of diagnosing the presence of abnormal respiratory function, said method comprising:
providing a patient with a device adapted for measuring inspired and expired carbon dioxide and oxygen and flow rate; ‘measuring the flow rate and partial pressures of carbon dioxide and oxygen in tidal breaths over a predetermined time period;
computing the ratio of carbon dioxide to oxygen in real-time;
visually plotting the computed ratios of carbon dioxide to oxygen; and
determining the presence of abnormal respiratory function based on the slope of predetermined portions of the plot of the computed ratios of carbon dioxide to oxygen.

20. The method of claim 19, wherein the ratio of carbon dioxide to oxygen is plotted as a function of volume.

21. The method of claim 19, wherein the ratio of carbon dioxide to oxygen is plotted as a function of time.

22. The method of claim 19, wherein the step of determining the presence of abnormal respiratory function includes comparing the slope of predetermined portions of the plot of the computed ratios of carbon dioxide to oxygen to the slope of predetermined portions of the plot of the ratios of carbon dioxide to oxygen in normal and afflicted subjects.

23. A method of diagnosing the presence of abnormal respiratory function, said method comprising:
providing a patient with a device adapted for measuring inspired and expired carbon dioxide and oxygen and flow rate;
measuring inspired and expired carbon dioxide and oxygen and flow rate;
determining the start of inspiration and the start of expiration based upon predetermined absolute thresholds and the measured flow rates;
calculating the average inspiration time and expiration time over a predetermined period of time;
calculating the ratio of the average inspiratory time divided by the expiratory time over the predetermined period of time; and
displaying the calculated ratio of the average inspiratory time divided by the expiratory time over the predetermined period of time.

24. The method of claim 23, further comprising the step of displaying the calculated ratio of the average inspiratory time divided by the expiratory time in combination with previously measured ratios of average inspiratory time divided by the expiratory time in normal and afflicted subjects.

25. The method of claim 23, further comprising the step of determining the presence of an airways obstruction based on the difference between the calculated ratio of the average inspiratory time divided by the expiratory time and the previously measured ratios of average inspiratory time divided by the expiratory time in normal and afflicted subjects.
